(19) United States
(12) Patent Application Publication
(10) Pub. No.: US 2005/0261654 A1
(43) Pub. Date: Nov. 24, 2005

(54) Method and apparatus for warming accessories for diapers and the like

(76) Inventor: Paul J. Swanson, Los Altos, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 10/850,468

(22) Filed: May 21, 2004

(51) Int. Cl. A61F 13/15

(52) U.S. Cl. 604/385.06

(57) ABSTRACT

In some illustrative embodiments, a disposable garment is provided, such as, e.g., a disposable diaper that is combined with a pouch (the terminology pouch is defined herein as encompassing a pocket, a container, an enclosure, a compartment, a housing and/or the like) that contains one or more clean-up wipes and/or other contents (herein referred to as diaper accessories). Preferably, the pouch is substantially moisture-proof (i.e., to retain internal moisture content and/or to inhibit external moisture entry). Preferably, the pouch includes an insulative layer on at least one side to help retain heat within said pouch or pocket. In the preferred embodiments, the pouch operates as a warming pouch. In various embodiments, the pouch can be used to contain a variety of accessories and, most preferably, a variety of diaper accessories, such as, by way of example, wipes, lotions, topical medications and/or powders.

### BACKGROUND

1. Field of the Invention

The present invention relates to diapers and diaper accessories, such as, e.g., wipes, lotions and/or other skin care accessories and/or the like and, certain of the preferred embodiments provide improved systems and methods for warming diaper accessories.

2. Discussion of the Background:

In modern society, it is well understood, and often the subject of humor and commentary, that diaper changing can be a complicated and difficult activity. Nevertheless, it is an activity that virtually every parent in our society must confront. Typically, diaper changing routines (whether employed by parents, care givers, medical professionals and/or the like or for infants, adults and/or the like) involves the cleanup of both a) soiled diapers and b) the wearer of the soiled diaper.

While substantial advances have been made in relation to the handling of the soiled diapers themselves, there remains a substantial need for improved systems and methods in relation to the handling of the wearer of the soiled diaper. Often, the wearer's body (e.g., the wearer's buttocks and surrounding regions, some of which are particularly sensitive to temperature, etc.) is cleaned using moistened wipes. However, the moistened wipes are typically stored at room temperature. Room temperature, which is typically between about 65-80 degrees Fahrenheit is substantially lower than the body temperature of the average wearer, which, e.g., for humans is typically about 98.6 degrees Fahrenheit. Upon contact of the moistened wipes against the body of the wearer, a substantial amount of heat transfer occurs, such as, e.g., via conductive heat transfer. While ambient air temperature is similarly at about 65-75 degrees Fahrenheit, the amount of heat transfer to the air is much more limited.

As a result, moistened wipes function to draw heat from the body of the wearer. This heat withdrawal can be quite uncomfortable, shocking and cold to a wearer. In fact, this heat dissipation may lead to other problems, such as, e.g., helping the patient to become chilled. This chilling can be exacerbated by the fact that during cleaning, the wearer's clothes are at least partially removed. Thus, a wearer that is initially insulated by clothing, usually has that insulation at least partly removed and is then further subject to chilling. This chilling can be particularly problematic when a wearer is already or is becoming sick. In addition, this chilling can be particularly problematic when a wearer is hospitalized, anesthetized and/or immobilized, such that the wearer's ability to retain body temperature may be diminished. In hospitals, for example, great lengths are taken to avoid hypothermia or chilling of patients, such as, e.g., providing specialized hot-air warming blankets and/or the like. Applying chilling materials against the bodies of wearers, especially after at least partial removal of insulating clothing or covering can, thus, be problematic in a variety of contexts.

While electronic warming devices that plug-in to electrical power sources are available for the pre-warming of wipes, these electronic warming devices have some notable deficiencies. First, these warming devices are cumbersome. They are typically rather heavy and not amenable to quick and easy transport. Second, they typically must be plugged into external power sources provided by public power utilities. Thus, they typically have restricted use locations. Third, they require additional steps for use. For example, a user must pre-fill the warmer and must pre-plan the location and placement of the warmer in relation to the user's activities at any given time. However, as is known by most parents and caregivers, any additional burdens to such parents and caregivers can be very difficult. Parents and caregivers typically have busy and hectic schedules and using such devices can be too difficult, especially at the time of changing of an infant or the like. Fourth, they may be somewhat expensive to purchase and/or to operate.

Thus, while the introduction of disposable diapers has offered significant advantages related to diaper handling and the relatively recent introduction of pre-packaged clean-up wipes has offered some limited advantages in relation to wearer clean-up, there remains a continued need for improved methods and systems, especially in the context of cleanup of a wearer.

There are a number of existing patents that are directed to wipes-containing-pockets integrated in diapers. However, these existing patents have a variety of limitations and deficiencies. Among other things, they do not contemplate, inter alia, overcoming the problems related to the chilling effects of wipes, much less how construct pockets to overcome such problems. The preferred embodiments of the present invention provide substantial advances over such existing systems and methods, such as, e.g., that described in the following patents, the entire disclosures of which are each incorporated herein in their entireties: (1) U.S. Pat. No. 6,475,204 entitled Diaper Construction, depicting a diaper in which a “baby wipe is contained within a cavity of a sealed pocket member provided within the diaper that is accessible only by tearing an access opening through an outer layer of the diaper and then rupturing a wall of the pocket to gain access to the baby wipe” (see Abstract); U.S. Pat. No. 6,454,748 entitled Diaper With A Pocket, depicting a diaper with “a pocket or pouch on the back of the diaper” (see column 4, lines 48-49); U.S. Pat. No. 4,964,859 entitled Diaper with Integral Changing Pad and Disposal Container, depicting a diaper in which a “changing pad and a disposable container means 20 is secured to the outer surface 14 of the diaper 10 so as to overlie the child's [buttocks] as shown in FIG. 1 until such time as the towelette is needed for wiping the skin” (see column 3, lines 18-20). While a variety of pockets are known in the art, none of the background art contemplates overcoming the chilling effect of such wipes, nor the construction of such pockets on a diaper in a manner to, inter alia, overcome these chilling effects.

Thus, while a variety of systems and methods have been known, prior to the present invention, there was a substantial need (although this need was not known or appreciated to those in the art) for more convenient methods of handling wipes and/or other diaper accessories and/or of warming wipes and/or other diaper accessories.

### SUMMARY OF THE INVENTION

The preferred embodiments of the present invention can significantly improve upon existing methods and/or apparatuses.

According to some embodiments, a method for warming at least one diaper accessory includes: providing a diaper with a pouch containing at least one diaper accessory; and locating the pouch proximate a heat source that raises the temperature of the at least one diaper accessory. In some embodiments, the at least one diaper accessory includes diaper wipes. Preferably, the diaper wipes include a plurality of moistened fabric sheets. In some embodiments, the at least one diaper accessory includes lotion, such as, e.g., for the moisturizing of skin, for the treatment of skin rashes, having antibacterial properties, having antifungal properties and/or the like. In some embodiments, the locating the pouch proximate a heat source that raises the temperature of the at least one diaper accessory includes locating the pouch proximate expelled urine content of the wearer to facilitate conductive heat transfer between the expelled urine content and an interior of the pouch. In some embodiments, the locating the pouch proximate a heat source that raises the temperature of the at least one diaper accessory includes locating the pouch proximate the wearer to facilitate conductive heat transfer between the wearer and an interior of the pouch.

In some embodiments, the temperature of the at least one diaper accessory while in the pouch is raised to greater than 85 degrees Fahrenheit, or, more preferably, to greater than 90 degrees Fahrenheit, or, more preferably, to greater than 92 degrees Fahrenheit, or, more preferably, to greater than 94 degrees Fahrenheit, or, more preferably, to greater than 96 degrees Fahrenheit, or, more preferably, to greater than 98 degrees Fahrenheit, or, more preferably, to proximate a body temperature of the wearer.

In some embodiments, the method further includes providing the pouch with a wearer-side conductive wall and with an environment-side insulated wall so as to facilitate conductive heat transfer from the wearer to the pouch and to retain conducted heat within the pouch.

According to some other embodiments, a warming pouch for at least one internal diaper accessory includes: a pouch containing at least one diaper accessory; the pouch including a wearer-side conductive wall that facilitates conductive heat transfer from a diaper wearer to the pouch; and the pouch including an environment-side insulated wall which facilitates retention of conducted heat within the pouch. In some embodiments, the pouch further includes an adhesive backing along the wearer-side conductive wall for adhering to an external surface of a diaper. In some embodiments, the pouch further includes an adhesive front along the environment-side insulated wall for adhering to an interior surface of a diaper.

According to some other embodiments, a combination is provided that includes: a) a warming pouch for at least one internal diaper accessory including: a pouch containing at least one diaper accessory; the pouch including a wearer-side conductive wall that facilitates conductive heat transfer from a diaper wearer to the pouch; and the pouch including an environment-side insulated wall which facilitates retention of conducted heat within the pouch; and b) a diaper upon which the pouch is mounted.

According to some other embodiments, a method of warming a skincare product includes: providing a pouch containing at least one skincare product; and adhering the pouch proximate to a source of heat emanated by the body of an individual. In some embodiments, the skincare product includes a skin lotion, and, in some embodiments, the skincare product includes wipes. In some embodiments, the method further includes adhering the pouch to the skin of the body of the individual with detachable adhesive.

According to some other embodiments, a method of educating consumers regarding the warming of at least one diaper accessory includes: a) providing a pouch for containing at least one diaper accessory on a diaper in a manner to enable heat transfer from a wearer of the diaper to said pouch; and b) informing consumers that the at least one diaper accessory inside said pouch can be warmed by the wearer's body heat when on the diaper in a manner to enable heat transfer from a wearer of the diaper to said pouch. In some embodiments, said informing includes commercial advertising to consumers (such as, e.g., via television, via newspaper, via Internet, via brochures, via packaging printing and/or other materials sold along with the product and/or the like). In some embodiments, said providing a pouch includes providing the pouch pre-attached to a diaper to the end consumer. In some embodiments, said providing a pouch includes providing a pouch separate from a diaper for attachment to the diaper by the end consumer.

The above and/or other aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### BRIEF DESCRIPTION OF THE DRAWINGS

The preferred embodiments of the present invention are shown by a way of example, and not limitation, in the accompanying figures, in which:

FIG. 1(A) is a front view of some illustrative embodiments demonstrating placement warming pouches on diapers according to some preferred embodiments;

FIG. 1(B) is a side view of the illustrative embodiments shown in FIG. 1(A) taken from the right side of FIG. 1(A) demonstrating placement warming pouches on diapers according to some preferred embodiments;

FIG. 2 is a side view of some illustrative embodiments showing an infant in a reclined position upon a changing pad while adorning a diaper with at least one integrated warming pouch;

FIGS. 3(A)-3(D) are views showing structure of warming pouches according to some illustrative and non-limiting embodiments of the invention, wherein FIG. 3(A) shows a side view of the illustrative warming pouch, FIG. 3(B) shows a cross-sectional front view of the illustrative warming pouch, FIG. 3(C) shows an end view of the illustrative warming pouch, and FIG. 3(D) shows a rear view of the illustrative warming pouch;

FIGS. 4(A)-4(B) are views showing structure of warming pouches according to some other illustrative and non-limiting embodiments of the invention, wherein FIG. 4(A) is a cross-sectional side view depicting illustrative warming pouch features according to some illustrative and non-limiting embodiments and FIG. 4(B) is a side view taken from the right side of FIG. 4(A);

FIGS. 5(A)-5(C) are perspective side views showing illustrative embodiments employing temperature indicators for, inter alia, indicating temperatures within the warming pouch;

FIG. 6 is a cross-sectional side view showing illustrative embodiments demonstrating illustrative placement of warming pouches, e.g., proximate expelled liquid urine content within a diaper and/or proximate the wearer's (such as, e.g., an infant's) point of urine discharge;

FIG. 7 is a cross-sectional, partial side view showing illustrative embodiments demonstrating illustrative placement of warming pouches, e.g., proximate the body of a wearer (such as, e.g., an infant);

FIGS. 8(A)-8(B) are cross-sectional, partial side views showing illustrative embodiment demonstrating illustrative interrelationships and heat-transfer between a wearer (such as, e.g., an infant) and illustrative warming pouches according to some illustrative embodiments, wherein FIG. 8(A) shows interrelationships and heat-transfer for some embodiments in which warming pouches are placed proximate expelled liquid urine content within a diaper and FIG. 8(B) shows interrelationships and heat-transfer for some embodiments in which warming pouches are placed proximate the body of the wearer;

FIG. 9 is a cross-sectional side view depicting some illustrative embodiments employing an activatable heat source, such as, e.g., a chemically activated heat source, a pressure activated heat source, an electrically activated heat source and/or the like; and

FIG. 10 is a front view showing illustrative embodiments in which a user has a pouch adhered to the user's skin for accommodating skin lotions and/or other pouch contents.

### DETAILED DESCRIPTION OF THE PREFERRED EMBODIMENTS

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

Introduction to the Preferred Embodiments

In some illustrative embodiments, a disposable garment is provided, such as, e.g., a disposable diaper that is combined with a pouch (the terminology pouch is defined herein as encompassing a pocket, a container, an enclosure, a compartment, a housing and/or the like) that contains one or more clean-up wipes and/or other contents (herein referred to as diaper accessories). Preferably, the pouch is substantially moisture-proof (i.e., to retain internal moisture content and/or to inhibit external moisture entry). Preferably, the pouch includes an insulative layer on at least one side to help retain heat within said pouch or pocket. In the preferred embodiments, the pouch operates as a warming pouch. In various embodiments, the pouch can be used to contain a variety of accessories and, most preferably, a variety of diaper accessories, such as, by way of example, wipes, lotions, topical medications and/or powders.

In preferred embodiments, the pouch can be adhered to a desired location on a diaper. Preferably, the pouch includes a re-attaching adhesive such that a consumer or user can freely attach, remove and re-attach the pouch at the desired location(s) on a diaper and/or on other locations which may or may not be in the proximity of the diaper.

In various embodiments, moistened wipes, lotions and/or the like can be stored in different ways, such as, e.g., by attaching the pouch directly to a particular region of the diaper directly, directly to the body of the wearer and/or to other appropriate locations such that the wipes, lotions and/or the like are warmed-up so as to be, inter alia, more comfortable for the wearer. Additionally, the preferred embodiments also enable the wipes, lotions and/or the like to be more readily accessible and conveniently located during use.

In various embodiments, a wipes warming package can be employed that is adapted to be adhered to a variety of locations for warming the wipes and/or for matters of convenience. Preferably, the warming pouch can be removed from a diaper and/or the like so as to re-stick the pouch somewhere else. For example, in some cases, the pouch can be re-stuck to a location proximate the parent, caregiver and/or the like during changing (such as, e.g., to a floor, table, their own body, etc.) to facilitate and/or accommodate cleaning of the wearer. This can be helpful for the changing of diapers for all wearers, including, e.g., infants, children, adults and elderly wearers.

According to some embodiments of the present invention, a unique infant, child, adult and/or elderly garment accessory can be provided that is convenient, practical and/or can simplify work of a parent, caregiver and/or the like.

According to some embodiments of the present invention, an accessory to a garment, such as, e.g., a disposable diaper or the like is provided that contains one or more sealed-in wipe(s) in a warmer pouch. Preferably, the pouch is easily removable, maintained desirably warm, and is readily usable (i.e., readily available for use at-the-scene of use) as needed.

According to a preferred embodiment of the invention, a garment is provided which includes a diaper having a substantially moisture-impervious pocket that is adherable thereto and that includes an insulation on one side, so as to contain, insulate and warm diaper accessories, when adhered to the diaper along with diaper accessories, such as, e.g., wipes therein. In some embodiments, the diaper and pouch can be manufactured together and distributed and sold together. In some embodiments, the pouch can be sold separately for integration and attachment to a diaper by an end consumer. In some embodiments, the pouch can be sold with accessories such as, e.g., wipes pre-stored therein. In some embodiments, the pouch can be re-usable, such that after removal from a diaper (i.e., which may, e.g., be disposed of), it can be attached to another new diaper and/or filled with new contents or accessories.

Among other things, a diaper or a garment according to the preferred embodiments can be highly advantageous, enabling, inter alia, diapers and wipes to be handled concurrently very easily, even when such are separately packaged and distributed products. Moreover, the preferred embodiments enable warm wipes or the like to be readily provided, without a need for separate heat sources, for separate manipulation, for separate power sources, etc.

In various other embodiments, a pouch could also be used to contain other products, such as, e.g., diaper rash lotions, skin creams and/or the like. In addition, in other embodiments, the pouch could include one or more other compartment for containing other items, such as, e.g., paper, notes regarding infant care to provide to a care giver, medicinal products, etc. In some embodiments, the pouch could contain a temperature indicator, such as, e.g., a temperature strip (discussed further below) to give a temperature indication or estimate. Among other things, the implementation of a temperature indicator can a) help confirm the temperature of wipes and/or b) help in early identification of health problems (such as, e.g., potential illness resulting in temperature change, potential over bundling or insulation of an infant in clothing or blankets, potential other problems resulting in raising of body temperatures, etc.).

Discussion of the Illustrated Embodiments

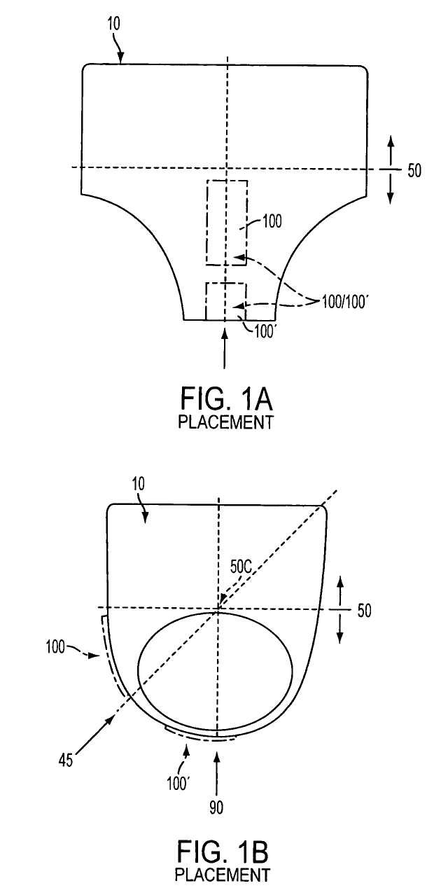

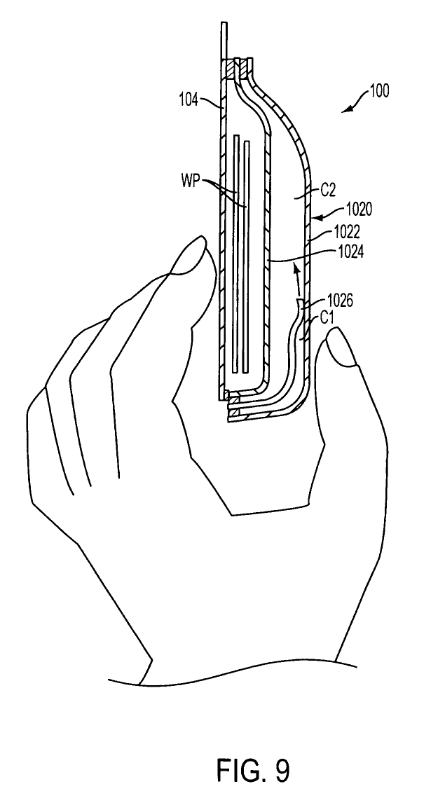

FIGS. 1-10 show some illustrative embodiments which, as discussed above, are merely illustrative and do not limit the broad scope of the invention. Those in the art should recognize based on this disclosure that the various embodiments can be modified and/or combined and/or varied as needed depending on circumstances.

In this regard, FIG. 1(A) is a front view of some illustrative embodiments demonstrating placement of warming pouches on diapers according to some preferred embodiments. To facilitate reference, FIG. 1(B) shows a side view taken from the right side of FIG. 1(A). FIGS. 1(A) and 1(B) show that in some preferred embodiments, a diaper 10 (which may include any currently or later known diaper structures, including disposable diapers, reusable diapers, etc., such as, e.g., any diapers as described in the above-referenced patents incorporated herein by reference above) has a pocket 100 attached thereto at certain locations in a manner to enhance warming of the pocket during use.

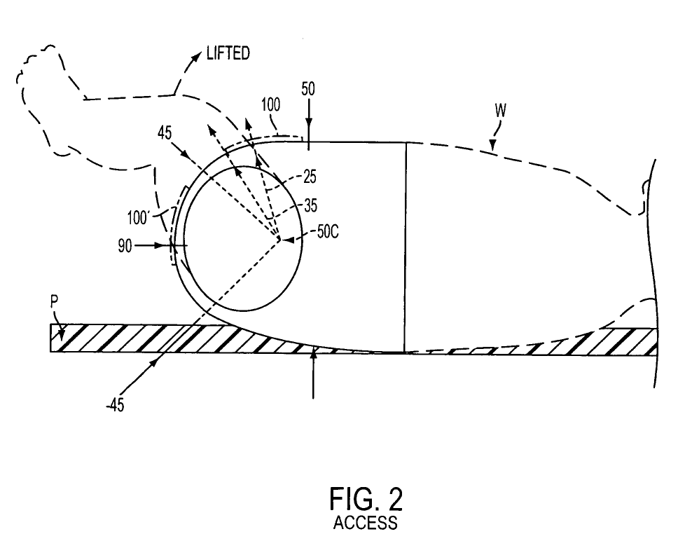

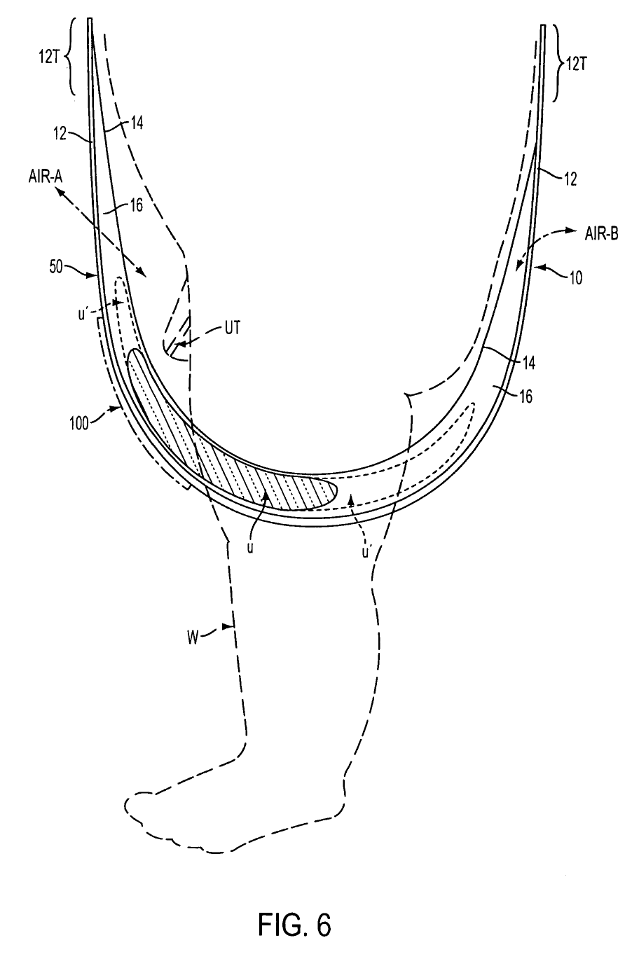

In this regard, as shown, at least one pocket 100 is preferably attached to the diaper 10 (e.g., upon an external surface of the diaper) at a location at least partly or, more preferably, entirely below the half-height line 50 of the diaper. The half-height line represents an imaginary line passing through the diaper half way up the total height of the diaper when in a use position as shown. In addition, while existing pockets are typically located upon the back of the diaper, in the preferred embodiments, such as, e.g., shown in FIGS. 1(A)-1(B), the pocket is preferably located proximate a front side of the diaper 10. Among other things, the placement of the pocket in such a location enables, e.g., as shown in FIGS. 2 and 6, the pocket 100 to be located within a region that can be further warmed due to, inter alia, proximity to expelled urine content within the diaper and/or proximity to the infant's legs as shown in FIG. 2. In this regard, the lower region of the diaper in the crotch region between the wearer's legs may in many cases have a higher heat retention due to the surrounding nature of the wearer's thighs and groin regions.

Additionally, as shown in FIG. 2, this region is also conveniently located during changing of an infant or the like. By way of example, the pouch 100 can be maintained on the diaper even until after the infant or the like is reclined upon a changing pad P as shown in FIG. 2 so as to enable easy and efficient access during use. Additionally, the pouch 100 can, thus, readily remain attached to the diaper and its heat source substantially until the time of use. While, in these preferred embodiments, the pouch is positioned toward a front side of the diaper, in some embodiments, the pouch can be positioned at other locations, such as, e.g., at a rear side of the diaper. By way of example, in some embodiments, the pouch can be positioned at about below the 50% line at the rear side of the diaper, or, more preferably, below about the minus 25 degree line (not shown), or, more preferably, below about the minus 35 degree line (not shown), or, more preferably, below about the minus 45 degree line shown in FIG. 2.

In some preferred embodiments, the pouch 100 is located such that at least a portion of or, more preferably, the entire pouch is located within a region substantially below an imaginary line 45 extending downward at a 45 degree angle from a center 50C of the 50% height of said diaper when adorned on a wearer. In some less preferred embodiments, the pouch 100 is located such that at least a portion of or, more preferably, the entire pouch is located within a region substantially below an imaginary line 35, shown in FIG. 2, extending downward at a 35 degree angle from a center 50C of the 50% height of said diaper when adorned on a wearer. In some even less preferred embodiments, the pouch 100 is located such that at least a portion of or, more preferably, the entire pouch is located within a region substantially below an imaginary line 25, shown in FIG. 2, extending downward at a 25 degree angle from a center 50C of the 50% height of said diaper when adorned on a wearer. In the illustrative example shown in FIGS. 1(A), 1(B) and 2, a pocket 100′ is shown as located entirely below the line 45. In some preferred embodiments, the pouch is located at least partly within a region extending across a bottom-most point 90 of said diaper. Among other things, in some preferred embodiments, the pouch 100 is selected so as to be situated proximate expelled urine when the diaper is filled with urine expelled from a wearer. In some preferred embodiments, the pouch can be situated so as to be located in front of or at least proximate a discharge of the wearer's urinary tract UT.

In some preferred embodiments, as shown in FIG. 6, the pouch is located so as to be substantially proximate expelled urine U when the diaper 10 is filled with urine U expelled from a wearer W. In this regard, FIG. 6 shows an illustrative diaper 10 which includes a substantially moisture-resistant outer layer 12, a substantially moisture penetrating inner layer 14 and a substantially moisture absorbent and/or retaining inner area 16. The inner area 16 can be filled with, e.g., a foam, an absorbent material, a fabric and/or the like material having high absorbance to retain and hold expelled urine U. In the illustrative example, the expelled urine U accumulates in a region proximate the discharge from the urinary tract UT to the diaper and/or proximate a forward/bottom region of the diaper. Accordingly, as shown, the pouch 100 is preferably located substantially proximate the discharged urine U. While the urine U is shown as within a pooled region U, it should be appreciated that the region can be located at other locations, at smaller locations, or at wider locations, such as, e.g., shown (as merely some illustrative and non-limiting examples) with the dashed extending lines U′ in FIG. 6.

In some preferred embodiments, the pouch 100 can be integrally mounted and attached to the noted positions on the diaper and sold in such a pre-attached condition to consumers. In other embodiments, the pouch 100 can be sold separately to consumers. Preferably, in such cases, the consumers are instructed as to appropriate positioning, such as, e.g., by providing pictures with the pouch 100 in desired positioning and/or by providing written instructions and/or the like. In some preferred embodiments, the user can locate the pouch 100 upon the diaper 10 based upon prior experience with the diaper type and the particular wearer (such as, e.g., to locate the pouch 100 proximate an estimated pooling location of urine). In this regard, the urine pooling location may have some degree of variability between wearers due to, e.g., gender (i.e., male discharge locations are often somewhat higher than that of female), wearer physical characteristics, wearer sleeping and/or other habits or positioning during wearing and/or the like.

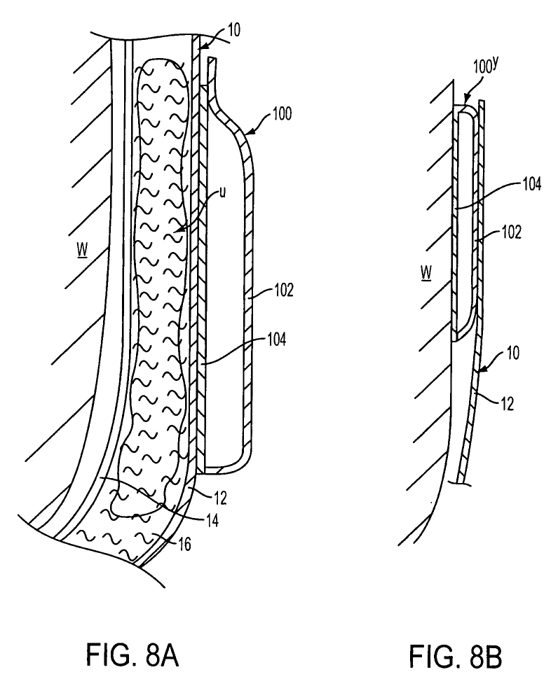

With reference to FIGS. 6 and 8, according to the most preferred embodiments, the pouch 100 is located in such a manner as to enhance heat transfer to said pouch and, in particular, conductive heat transfer to said pouch. In this regard, in preferred embodiments, the positioning of the pouch 100 is preferably selected so as to reduce the insulating factor of the diaper situated in between the wearer W and the pouch 100.

In this regard, conductive heat transfer can be estimated, in some examples, by the following formula.

$$\frac{Q}{t} = \frac{κ A ( {T}_{hot} - {T}_{cold} )}{d}$$

In this formula, Q is the heat transferred in time t, k is the thermal conductivity of the barrier, A is the area of the barrier, Tₕₒₜ and T_{cold} are the respective temperatures, and d is the thickness of the barrier. This formula helps to illustrate that the heat transfer to the pouch 100 depends appreciably upon the distance from the heat source and upon the thermal conductivity of the barrier. Accordingly, existing pouches located, inter alia, in undesirable positions resulted in having such pouches actually insulated from heat transfer due to a large distance d and a low thermal conductivity k. For example, placement of pouches on an upper/rear portion of a diaper can lead to typical cases in which urine content is distant from the pouch 100 and wherein the diaper 10 acts as an insulator (e.g., by virtue of the high insulative characteristics of absorbent materials in their non-absorbed conditions). The thermal conductivity of such non-absorbed state can approach that of, e.g., air, foam and/or other insulative materials. On the other hand, the thermal conductivity of such materials in an absorbed state can approach that of, e.g., water. As a result, there is a large degree of variation in conductive heat transfer achieved. For instance, the following Table 1, which is provided for illustrative purposes only, demonstrates that there may be a large variation in thermal conductivity as between absorbent material with pooled urine (which may be similar to, e.g., water) and such absorbent material in a non-absorbed state (which may be similar to, e.g., air or Styrofoam) in some examples.

TABLE 1
(Approximate Thermal Conductivity of Illustrative Materials)

| | Thermal conductivity | Thermal conductivity |
|---|---|---|
| Material | (cal/sec)/(cm² C./cm) | (W/m K)* |
| Aluminum | 0.50 | 205.0 |
| Water at 20 C. | 0.0014 | . . . |
| Fiberglass | 0.00015 | 0.04 |
| Styrofoam | . . . | 0.01 |
| Air at 0 C. | | |
| | 0.000057 | 0.024 |

As shown in FIG. 8(A), in some preferred embodiments, the pouch 100 is preferably conveniently located outside of an outer wall 12 of a diaper 10, but at the same time is preferably strategically positioned proximate the body-heated urine pool U within the inner area 16. As shown in FIG. 8(A), in some preferred embodiments, the distance d in the above-noted conductive heat transfer formula is very small and can be less than a few millimeters, such as, e.g., less than about 5 millimeters, or, more preferably, less than about 3 millimeters, or, more preferably, less than about 2 millimeters. As shown, the distance d to the interior of the pouch can be, e.g., about the same or the same as the thickness of the wall 12, an inside wall 104 of the pouch and any adhesive or the like there-between.

In some preferred embodiments, as shown in FIG. 8(A), the conductive properties of the walls 12 and 104 combined may be approximately equal to that of a thin plastic sheet (such as, e.g., a thin polypropylene sheet of less than 1 mm thickness, or, more preferably of less than 0.5 mm thickness). In some preferred embodiments, the outer wall 102 of the pouch is insulated so as to inhibit heat transfer there-through. In some preferred embodiments, the conductive heat transfer possible through the wall 104 is more than 2 times that of the conductive heat transfer possible through the wall 102, or, in some embodiments, more than about 5 times that of the conductive heat transfer possible through the wall 102, or, in some embodiments, more than about 10 times that of the conductive heat transfer possible through the wall 102. Similarly, the wall 102 is preferably more than two times as insulative as the barrier between the urine U and the pocket interior, or, in some embodiments, more than five times as insulative, or, in some embodiments, more than 10 times as insulative, or, in some embodiments, more than 20 times as insulative or more (wherein such insulative properties can be determined based on, e.g., the respective R-Values, such as, e.g., based on square feet x hour x degrees F. over BTU).

In considering the heat transfer in a diaper environment, it is notable that diapers are often constructed for some “breathability” such that air within the diaper is preferably not stagnated. Among other things, this may help to keep the child or the like cooler (i.e., to prevent overheating), to prevent build-up of moisture (e.g., to prevent discomfort due to bacteria build-up, skin irritation, rashes, etc.). Towards this end, diapers often provide some convective airflow through diaper wall to-and-from the interior. Typically, this airflow reduces internal temperature using convective air flow principles. By way of example, FIG. 6 depicts illustrative air flow paths AIR-A and/or AIR-B through the diaper 10.

Among other things, this air flow can substantially reduce heat transfer between the body of the wearer W to any pocket or pouch on a diaper. In this regard, convection can involve heat transfer due to mass motion of, e.g., air when the heated air is caused to move away from the source of heat. Convection can further limit the temperature applied to a wipe(s) contained in a warming pouch. However, when wipes are strategically positioned near urine pool U, even if air flow is between urine pool and the wearer W (e.g., child), the urine pool U may retain heat longer and, hence, retain temperature of wipes longer. In this regard, urine U is discharged from the wearer at body temperature, and, while separated within the diaper so as to create an airspace between the urine and the body of the wearer in some embodiments, the heat retention of fluid is substantially longer than that of air. In some preferred embodiments, substantial conductive heat transfer occurs between the urine U and the pouch 100. Preferably, a significant amount of this conductive heat transfer occurs substantially without an air space between the urine pool U and the pouch 100.

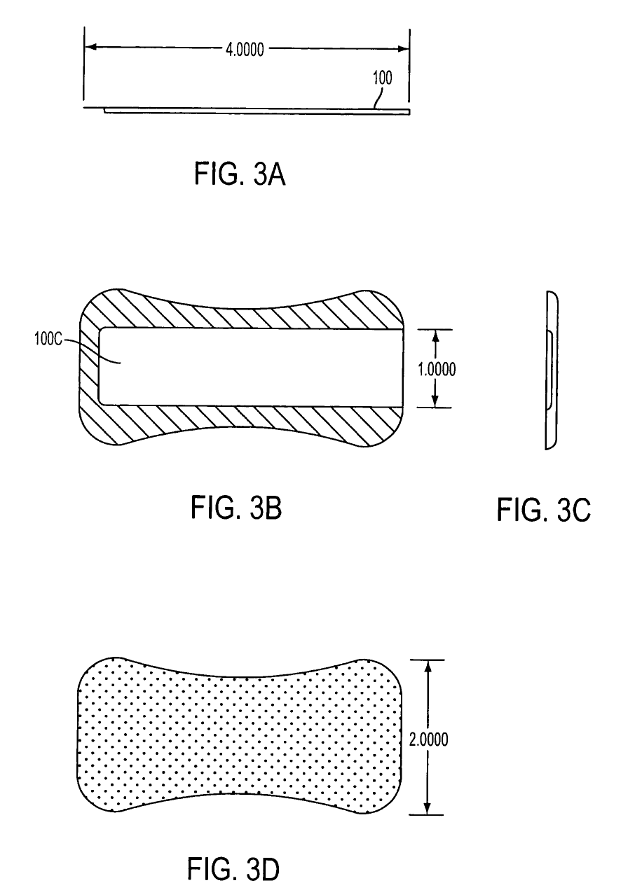

FIGS. 3(A)-5 show some illustrative examples of pouches 100 that may be employed in some embodiments. In this regard, FIGS. 3(A)-3(D) show an illustrative pouch according to some preferred embodiments of the invention. The embodiment shown in FIGS. 3(A)-3(D) are particularly advantageous when used, inter alia, placed in a crotch region between a wearer's legs, such as, e.g., shown by the pouch 100 in FIG. 2. As shown in FIG. 3(A), the pouch 100 can have a length 4 which may be, e.g., about 3 to 5 inches, or in some illustrative examples as shown, about 4 inches in length and with a narrow width of less than about 1/3 inch, or, more preferably, less than about 1/4 inch, or, more preferably, less than a few millimeters. As shown in FIG. 3(B), the pouch may, in some cases, have an hour-glass configuration to follow the contour along the crotch region between the wearer's legs. As shown, the pouch can have a width 2 which may be, e.g., about 1 to 3 inches, or in some illustrative examples as shown, about 2 inches in width and with a narrowed region proximate the middle thereof as shown in FIG. 3(B). As shown in FIG. 3(C), the pouch 100 can include an interior chamber 100 C having a width of about ½ or so, for example, of the width of the pouch and having a depth of about ½ or so, for example, of the depth of the pouch. With reference to FIG. 3(C), the pouch preferably includes a thicker region on an exterior side thereof (e.g., shown at the right side of the cross-section shown in FIG. 3(C)) and a narrower region on the interior side thereof (e.g., shown at the left side of the cross-section shown in FIG. 3(C)). In this manner, e.g., the exterior side can include, in preferred embodiments, insulative materials to retain heat while the interior side can include, in preferred embodiments, materials selected to enable conduction of heat to the pouch.

In some preferred embodiments, the pouch 100 shown in FIGS. 3(A)-3(D) can include an adhesive on the rear-side thereof (e.g., a side facing to the left in FIG. 3(C)). In some preferred embodiments, a plurality of pouches 100 can be sold, in a pre-filled condition—such as, e.g., with wipes, lotions, and/or other diaper accessories therein—to end consumers or end users. In such cases, the pouches can be, e.g., packaged in groups for distribution to retailers and sold in packs of a plurality of pouches, such as, e.g., in packs of 10, 20, 30, 40 and/or the like. In some embodiments, the pouches can be sold in detached states from diapers, but in sets or paired-up with diapers (such as, e.g., shrink wrapping a bundle of diapers and a bundle of pouches together for co-sale to consumers).

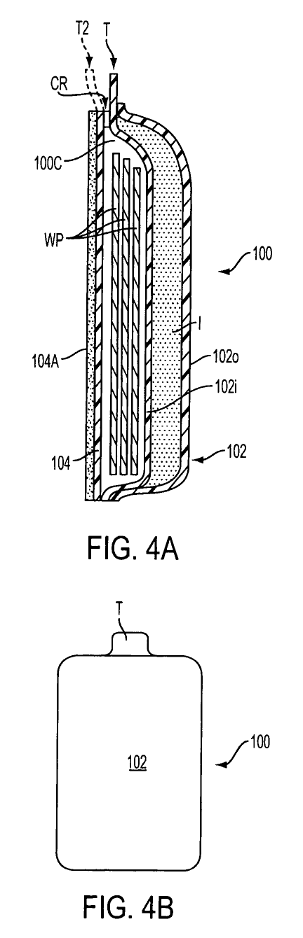

Reference is now made to FIGS. 4(A)-4(B) which show warming pouches according to some other illustrative and non-limiting embodiments of the invention, wherein FIG. 4(A) is a cross-sectional side view depicting illustrative warming pouch features according to some illustrative and non-limiting embodiments and FIG. 4(B) is a side view taken from the right side of FIG. 4(A). In these illustrative embodiments, the pouch 100 includes an inside wall 104 and an outside wall 102. The inside and outside walls are preferably formed of moisture resistant or moisture proof materials, such as, e.g., elastomers, plastics and/or the like. In preferred embodiments, the materials are also substantially flexible such that the pouch 100 can relatively freely flex and/or bend along with movement of the wearer. As shown, the inside of the wall 104 preferably includes an adhesive layer. Although not shown, a cover layer can be adhered over the adhesive layer 104A to protect the same until use (e.g., at which time the cover layer could be removed and discarded). Preferably, the interior of the pouch 100 defines a substantially moisture-sealed compartment 100C, which can contain, e.g., a plurality of moisturized wipes WP (i.e., three are shown in the illustrated and non-limiting embodiment). Preferably, the pouch can be manually opened to enable access to the compartment 100C. By way of example, in some embodiments, a manual pull tab T can be integrally formed in the outer wall 102 which can be pulled to separate the wall 102 from the wall 104.

In some embodiments, the wall 104 can also include a pull tab T2. By way of example, in use, the user can initially apply the entire pouch 100 onto a proper region of a diaper. Then, during cleaning, the user can grasp both tabs T and T2, concurrently, and can peal the pouch 100 from the diaper. Then, the user can grasp both tabs T and T2 individual and open the compartment 100C so as to access the wipes WP. It should be understood based on this disclosure that any other structure for enabling the pouch to be opened can be employed. For example, the pouch 100 could be torn open, cut open, unzipped and/or otherwise opened in various embodiments. As shown, the connecting region CR between the walls 102 and 104 at which the pouch 100 would be opened can include any appropriate connection structure such as, e.g., a heat seal, a pressure sensitive adhesive seal, a pressure seal, a zip-lock seal, a snap connection and/or any other appropriate connection.

As also shown in FIG. 4(A), the pouch 100 can also be formed such that the outer wall includes an inside wall portion 102i, an outside wall portion 102o and an internal insulation material 1, such as, e.g., foam, filler material, one or more air space(s) and/or the like so as to enhance heat retention.

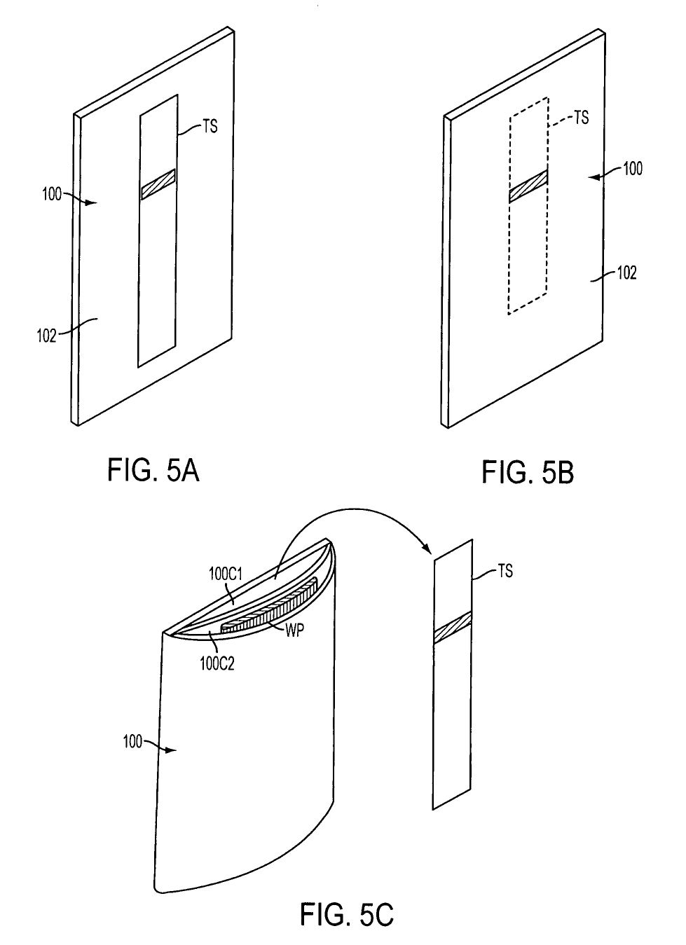

Reference is now made to FIGS. 5(A)-5(C) which are perspective side views showing illustrative embodiments employing temperature indicators for, inter alia, indicating temperatures within the warming pouch. In this regard, in some embodiments, the pouch can include a temperature indicator, such as, e.g., a thermometer. While any type of thermometer device could be employed, in some preferred embodiments, the thermometer device includes a temperature strip (similar to that used on foreheads) to give a temperature estimate. This can be used, e.g., to a) confirm temperature of wipes and to b) help in early identification of health issues (such as, e.g., whether illness resulting in temperature change occurs, whether the caregiver has inadvertently over-bundled the infant, resulting in raising of body temperatures, etc.). By way of example, in some embodiments, a temperature strip like that of the FEVERSCAN temperature strip manufactured by Hallerest Products, 1820 Pickwick Lane, Glenview, III., 60025 can be employed. Illustrative temperature strips can include, e.g., plastic strips containing heat-sensitive liquid crystals that change color to indicate the temperature. Temperature strips are particularly ideal since they are typically flexible and flat and can, thus, be readily integrated into such a pouch 100. FIGS. 5(A)-5(C) show three illustrative methods of such integration. First, FIG. 5(A) shows the integral integration of a temperature strip TS in the outer wall 102 of the pouch 100. In this manner, the temperature strip can be easily viewed. Second, FIG. 5(B) shows the locating of a temperature strip TS inside of the pouch 100, while the outer wall 102 is made of a sufficiently transparent or translucent material to enable the strip TS (shown in dashed lines due to show its being located inside of the pouch 100) to be observed there-through. Third, FIG. 5(C) shows another embodiment in which the temperature strip TS is located inside of the pouch 100 upon the diaper that is adorned by the wearer and is removed during use by the consumer. In some embodiments, the temperature strip could be contained in the same compartment 100C as the wipes WP and/or the like contents, while in other embodiments, the temperature strip could be contained in a separate compartment, such as, e.g., the compartment 100C1 shown in FIG. 5(C), while the wipes WP and/or the like are contained in another compartment, such as, e.g., the compartment 100C2 as shown. In this latter embodiment, the temperature strip TS may, inter alia, be retained for future use after discarding the pouch 100 (if the pouch is to be disposable or disposed of), removed for taking of forehead and/or other temperature readings and/or the like.

While in some preferred embodiments, the wipes or other contents are preferably heated by the body heat of the wearer, in other embodiments, the wipes can be heated via other means. However, the other means preferably does not require the maintenance and/or use of another external device and/or the connection to a utility company power source, such as, e.g., by plugging an electrical power chord into an electric power outlet. By way of example, in some embodiments, the pouch 100 could be attached to another source of heat such as, e.g., upon a solar heated surface (such as, e.g., a car dashed board), upon the skin of any individual or animal, upon a hot window or hot surface, upon a heated lamp and/or the like. In preferred embodiments, the pouch 100 can be removably attached to a diaper and detachably placed upon any appropriate surface as desired during use.

In some other embodiments, the pouch 100 can also and/or alternatively include its own heat source, such as, e.g., using heat from a chemical reaction, from a battery powered mechanism and/or from other appropriate means. By way of example, the pouch 100 could include an outer enclosure 1020 formed with inner and outer walls 1024 and 1022, respectively, and which is initially separated into two chambers C2 and C1 by a separation wall 1026. During use, a user can squeeze the region adjacent chamber C1 such that the strip 1026 separates from the wall 1022 causing contents of the chambers C1 and C2 to mix and, thus, to create heat (i.e., based on special pre-selection of materials). Additionally, in some embodiments, the pouch could include a compartment similar to C2 filled with a liquid such as, e.g., a sodium acetate solution that can be used to generate heat upon the pressing of a metal activation disc or the like to trigger crystallization changing the solution from a solid to a liquid, such as, e.g., seen in HEALING HEAT PACKS by Natural Wellness Company, Montgomery, N.Y. The latter examples may be particularly advantageous since the heating source can be re-used, such as, e.g., by placement of the pouch 100 within boiling water to return the contents to a liquid form, enabling reuse to heat other wipes or the like at a later time. Whether or not this latter principle is employed on a wipes pouch attached to a diaper, this would be advantageous in conjunction with any type of wipes pouch, enabling the wipes to be promptly heated on demand with external heat sources.

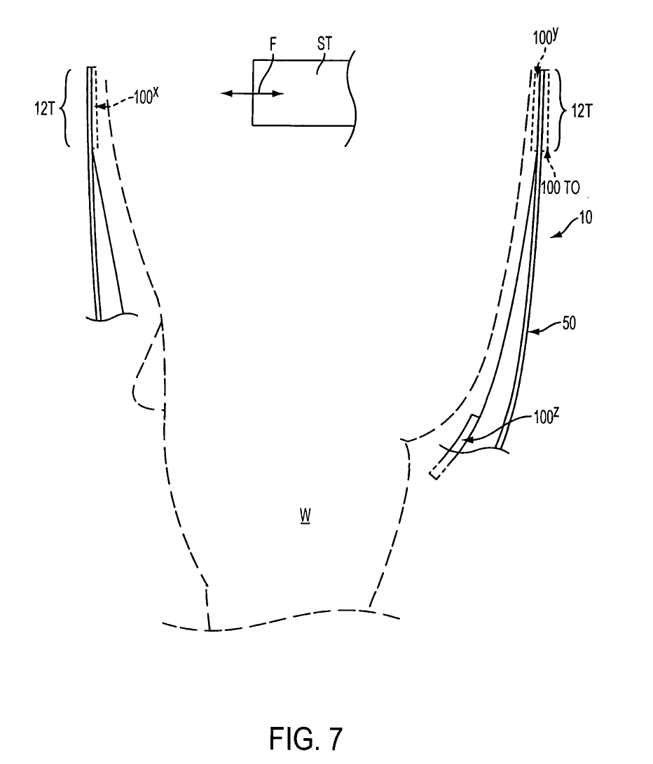

While many of the embodiments described herein-above relate to the attachment of a pouch on the exterior surface of a diaper, in some embodiments, a pouch can be attached at other locations of a diaper. Nevertheless, external attachment to a diaper can be particularly advantageous from the standpoints of, inter alia, ease of use, ease of access, cleanliness, etc. However, in some instances, it can be advantageous to have pouches attached to other locations. By way of example, FIG. 7 is a cross-sectional, partial side view showing illustrative embodiments demonstrating illustrative placement of warming pouches, e.g., adjacent or proximate the body of a wearer (such as, e.g., an infant). FIG. 7 shows a diaper 10 that can be substantially similar to, e.g., the diaper shown in FIG. 6. However, in the embodiments shown in FIG. 7, the pouches 100^{x}, 100^{y}, 100^{z} are located inside the diaper. In these examples, the pouches 100^{x} and 100^{y} have some notable advantages in that they can be located substantially adjacent and more readily maintained against the skin of the wearer. As a result, the conduction between the wearer W and the pouch 100 can be facilitated to help ensure proper warming of the pouch contents. In some preferred embodiments, the pouches are located within an upper region 12 T that is at or adjacent the upper end of the diaper. This region can include, e.g., the top few inches, e.g., the top 2 inches or less of the diaper, and may include, e.g., a region of the diaper commensurate with tying straps, elastic members and/or the like which helps constrict the diaper so as to be retained snuggly against the wearer. Placement within this region can, thus, help ensure that the pouch is snuggly held against the wearer, can help ensure that the pouch is away from excretion contents so as to facilitate sanitary handling (notably, this region 12T often does not require any absorbent material in diapers and, thus, may be particularly thin in some cases).

FIG. 8(B) shows a close-up view of a pouch similar to the pouch 100^{y} shown in FIG. 7. Although not depicted in FIG. 8(B), in this embodiment, the wall 104 can advantageously be made very thin and adapted to enhance heat conduction there-through, while the wall 102 can, if desired, be made to include an insulative structure similar to the wall 102 shown in, e.g., FIG. 4(A).

As shown in dashed lines in FIG. 7, in some alternative embodiments, a pouch 100-TO can be attached within the region 12T above the insulative material of the diaper in a manner to be snugly held against the body of the infant, but rather than be directly held against the body of the infant, being snugly held outside of a thin wall of the diaper. In this cases, the pouch is preferably located substantially or, more preferably, entirely within the region 12T; and, the region 12T proximate the pouch preferably does not include insulative or absortive material; and, a snug fit is preferably achieved due to the application of tightening forces F (such as, e.g., due to one or more straps ST or by elasticity). In this manner, a higher heat conduction to the pouch 100 can be achieved. Here, the pouch 100-TO can be formed in a variety of ways and can include, e.g., a pouch similar to that shown in FIG. 4(A) in some preferred embodiments. While the pouch 100-TO is shown as being proximate a top rear of the diaper, it can be located in other positions along the top region 12T, such as, e.g., proximate the front of the diaper. As a result, in these embodiments, even if the pouch is mounted externally to the diaper, it can be substantially snugly held against the wearer, with minimal distance of separation from the wearer of about the thickness of the fabric of the diaper without substantially any insulation (such as, e.g., wherein the thickness of the diaper between the pouch and the wearer is well under 2 millimeters, or well under about 1 millimeters, or even well under about 0.5 millimeters, or even well under about 0.1 millimeters in some embodiments).

In some embodiments, a pouch according to some embodiments described herein can be attached directly to the skin of the user. For example, the pouch can include a detachable adhesive that can be used to attach the pouch to the skin of a user. In these embodiments, the contents of the pouch can readily be brought to a level close to the wearer's peripheral body skin temperature. In these cases, for example, the pouch could be attached to the skin of the wearer, such as, e.g., the wearer's buttocks, hip, back, stomach, arm, chest, thigh and/or the like as desired. Additionally, the pouch could also be attached to the buttocks, hip, back, stomach, arm, chest, thigh and/or the like of the parent, caregiver and/or the like. In this manner, the parent, caregiver and/or the like can have quick and easy access to the wipes at any time. Additionally, by attachment to the parent, caregiver and/or the like, any discomfort related to attachment, wearing and/or detachment of the pouch will not be caused to the wearer of the diaper. Thus, the comfort of the diaper wearer can be well maintained.

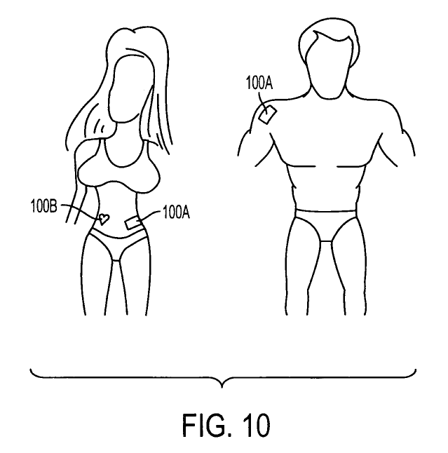

Notably, in some embodiments, a pouch according to the latter embodiment can be employed in contexts even outside of diaper usage. For example, a unique lotion or the like holding pouch could be provided that a user can readily carry as needed (such as, e.g., attaching to their shoulder or the like). By way of example, sun tan lotion, skin moisturizing lotion, sun block lotion and/or the like could be easily carried to a beach, pool, and/or during any other outside activity by an individual, without having to manually carry the same, put the same in clothing pockets and/or the like. This can be a very convenient way to ensure that individual's, who may, for example, be adorned in pocket-less swimwear can have appropriate lotions on hand (or, in actuality, “on body”). Thus, these latter embodiments can, if desired, provide one or both of the following advantages: a) facilitating handling of lotions or the like and/or b) keeping lotions or the like at a more comfortable temperature. In this regard, FIG. 10 is a front view showing some illustrative embodiments in which a user has a pouch adhered to the user's skin for accommodating skin lotions and/or other pouch contents. In these illustrative embodiments, the wearer can adorn the pouches in desired locations, such as, e.g., upon the user's hip (as shown upon a female user's hips at 100A and 100B), upon the user's shoulder (as shown upon a male user's shoulder at 100A) and/or the like. These embodiments may provide advantage that users may use such pouches as a means to attract attention to themselves, such as, e.g., placement on physically appealing body parts, such as, e.g., buttocks, hips, bellies, shoulders, etc. In some embodiments, in addition and/or alternatively to warming by body heat, the pouch could be warmed by, e.g., solar energy, such as, e.g., by having a color (e.g., black) that absorbs sunlight. In some embodiments, the pouch can include additional compartments for other items, such as, e.g., driver's licenses, money, change and/or the like. In some embodiments, the pouch can have exterior decoration, coloring, ornamentation and/or the like to enhance appeal. In some embodiments, the pouch can have a variety of configurations, such as, e.g., being formed in the shape of a particular image or object (such as, e.g., a plant or an animal [such as, e.g., a flower, a butterfly, a cat and/or the like], a pictorial character, a cartoon character, a television character, a product representative character [such as, e.g., a trademark character], dancing bear characters, a peace sign, etc.), a heart, a skull, a bottle, a tube and/or the like), a generally hour-glass configuration as shown in FIG. 3(B), a generally rectangular configuration as shown in FIG. 10, a round configuration and/or any other desired configuration. In some embodiments, the pouch can include printed indicia thereon, with, e.g., company logos, trademarks, slogans or the like, humorous statements printed thereon and/or other alpha-numeric indicia. Notably, pouches adorned in this manner could also be used to enable wearers to achieve ornamental tan-free regions (essentially creating, e.g., a “tattoo” resultant from a blocked region due to the pouch 100A, 100B, such as, e.g., in the shape of a heart 100B or the like).

Broad Scope of the Invention:

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” Means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited.

1. A method for warming at least one diaper accessory, comprising:
a) providing a diaper with a pouch containing at least one diaper accessory; and
b) locating the pouch proximate a heat source that raises the temperature of the at least one diaper accessory.

2. The method of claim 1, wherein said at least one diaper accessory includes diaper wipes.

3. The method of claim 1, wherein said diaper wipes include a plurality of moistened fabric sheets.

4. The method of claim 1, wherein said at least one diaper accessory includes lotion.

5. The method of claim 4, wherein said lotion is lotion for the moisturizing of skin.

6. The method of claim 5, wherein said lotion is lotion for the treatment of skin rashes.

7. The method of claim 5, wherein said lotion includes antibacterial properties.

8. The method of claim 5, wherein said lotion includes antifungal properties.

9. The method of claim 1, wherein said locating the pouch proximate a heat source that raises the temperature of the at least one diaper accessory includes locating the pouch proximate expelled urine content of the wearer to facilitate conductive heat transfer between said expelled urine content and an interior of said pouch.

10. The method of claim 1, wherein said locating the pouch proximate a heat source that raises the temperature of the at least one diaper accessory includes locating the pouch proximate the wearer to facilitate conductive heat transfer between said wearer and an interior of said pouch.

11. The method of claims 1, 9 or 10, further including raising said the temperature of said at least one diaper accessory while in said pouch to greater than 85 degrees Fahrenheit.

12. The method of claim 11, further including raising said the temperature of said at least one diaper accessory while in said pouch to greater than 90 degrees Fahrenheit.

13. The method of claim 11, further including raising said the temperature of said at least one diaper accessory while in said pouch to greater than 92 degrees Fahrenheit.

14. The method of claim 11, further including raising said the temperature of said at least one diaper accessory while in said pouch to greater than 94 degrees Fahrenheit.

15. The method of claim 11, further including raising said the temperature of said at least one diaper accessory while in said pouch to greater than 96 degrees Fahrenheit.

16. The method of claim 11, further including raising said the temperature of said at least one diaper accessory while in said pouch to either greater than 98 degrees or to proximate a body temperature of the wearer.

17. The method of claim 1, further including adhering said pouch to said diaper with an adhesive.

18. The method of claim 17, wherein said adhesive is a removable adhesive and further including removing said pouch from said diaper by virtue of said removable adhesive.

19. The method of claim 17, further including providing the pouch to a consumer, wherein said pouch includes an adhesive backing with a protective cover layer, and having said consumer remove said protective cover layer and place said pouch upon the diaper with said adhesive backing.

20. The method of claim 17, further including having a consumer locate the pouch upon said diaper.

21. The method of claim 20, further including having the consumer locate the pouch proximate expelled liquid urine content based upon the consumer's experience.

22. The method of claim 1, wherein said wearer is a human infant under 5 years of age.

23. The method of claim 1, wherein said wearer is a human or non-human wearer from the group consisting of infant wearers, mature wearers, elderly wearers, sick wearers or injured wearers.

24. The method of claim 1, further including providing said pouch with a wearer-side conductive wall and with an environment-side insulated wall so as to facilitate conductive heat transfer from the wearer to the pouch and to retain conducted heat within said pouch.

25. The method of claim 1, wherein said locating the pouch proximate a heat source that raises the temperature of the at least one diaper accessory includes locating at least a portion of the pouch within a region substantially below a 50% height of said diaper.

26. The method of claim 1, wherein said locating the pouch proximate a heat source that raises the temperature of the at least one diaper accessory includes locating the pouch entirely within a region below a 50% height of said diaper.

27. The method of claim 1, wherein said locating the pouch proximate a heat source that raises the temperature of the at least one diaper accessory includes locating at least a portion of the pouch within a region substantially below a downward 45 degree angle from a center of a 50% height of said diaper when adorned on a wearer.

28. The method of claim 1, wherein said locating the pouch proximate a heat source that raises the temperature of the at least one diaper accessory includes locating the pouch entirely within a region below a downward 45 degree angle from a center of a 50% height of said diaper when adorned on a wearer.

29. The method of claim 25, 26, 27 or 28, further including locating said pouch on a front side of said diaper.

30. The method of claim 25, 26, 27 or 28, further including locating said pouch on a rear side of said diaper.

31. The method of claim 1, wherein said locating the pouch proximate a heat source that raises the temperature of the at least one diaper accessory includes providing the heat source without the use of electrical energy supplied by a public power company facility.

32. The method of claim 1, wherein said locating the pouch proximate a heat source that raises the temperature of the at least one diaper accessory includes providing the heat source as heat from the wearer.

33. The method of claim 1, wherein said locating the pouch proximate a heat source that raises the temperature of the at least one diaper accessory includes providing the heat source as heat from a heat source attached to or within said pouch.

34. The method of claim 33, further including having a consumer manually activate said heat source when said at least one diaper accessory is to be warmed.

35. The method of claim 1, further including having an end user initially separately acquire said diaper and said pouch and subsequently attaching said pouch to an outside or an inside surface of said diaper.

36. A warming pouch for at least one internal diaper accessory, comprising:
a) a pouch containing at least one diaper accessory;
b) said pouch including a wearer-side conductive wall that facilitates conductive heat transfer from a diaper wearer to the pouch; and
c) said pouch including an environment-side insulated wall which facilitates retention of conducted heat within said pouch.

37. The pouch of claim 36, further including an adhesive backing along said wearer-side conductive wall for adhering to an external surface of a diaper.

38. The pouch of claim 36, further including an adhesive front along said environment-side insulated wall for adhering to an interior surface of a diaper.

39. In combination:
a) a warming pouch for at least one internal diaper accessory, comprising:
i) a pouch containing at least one diaper accessory;
ii) said pouch including a wearer-side conductive wall that facilitates conductive heat transfer from a diaper wearer to the pouch; and
iii) said pouch including an environment-side insulated wall which facilitates retention of conducted heat within said pouch;
b) a diaper upon which said pouch is mounted.

40. The combination of claim 39, wherein said pouch is located at least partly within a region substantially below a 50% height of said diaper.

41. The combination of claim 39, wherein said pouch is located entirely within a region below a 50% height of said diaper.

42. The combination of claim 39, wherein said pouch is located at least partly within a region substantially below a downward 45 degree angle from a center of a 50% height of said diaper when adorned on a wearer.

43. The combination of claim 39, wherein said pouch is located entirely within a region substantially below a downward 45 degree angle from a center of a 50% height of said diaper when adorned on a wearer.

44. The combination of claim 39, wherein said pouch is located at least partly within a region extending across a bottom-most point of said diaper.

45. The combination of claim 39, wherein said diaper is filled with urine expelled from a wearer and said pouch is located proximate said expelled urine.

46. The combination of claim 45, wherein conductive heat transfer occurs between said urine and said pouch.

47. The combination of claim 46, wherein a significant amount of said conductive heat transfer occurs without an air space between said urine and said pouch.

48. The combination of claim 39, wherein said pouch is adhered to said diaper with a removable adhesive.

49. A method of warming a skincare product, comprising:
a) providing a pouch containing at least one skincare product; and
b) adhering the pouch proximate to a source of heat emanated by the body of an individual.

50. The method of claim 49, wherein said skincare product includes a skin lotion.

51. The method of claim 49, wherein said skincare product includes wipes.

52. The method of claim 49, further including adhering the pouch to the skin of the body of the individual with detachable adhesive.

53. The method of claim 49, further including adhering the pouch to a garment at a position adjacent a source of urine expelled from the body of the individual.

54. The method of claim 49, further including providing the pouch with a wearer-side conductive wall that facilitates conductive heat transfer to the pouch, an environment-side insulated wall which facilitates retention of conducted heat within said pouch and with an adhesive along said wearer-side conductive wall.

55. A method of educating consumers regarding the warming of at least one diaper accessory, comprising:
a) providing a pouch for containing at least one diaper accessory on a diaper in a manner to enable heat transfer from a wearer of the diaper to said pouch; and
b) informing consumers that the at least one diaper accessory inside said pouch can be warmed by the wearer's body heat when on the diaper in a manner to enable heat transfer from a wearer of the diaper to said pouch.

56. The method of claim 55, wherein said informing includes commercial advertising to consumers.

57. The method of claim 55, wherein said providing a pouch includes providing the pouch pre-attached to a diaper to the end consumer.

58. The method of claim 55, wherein said providing a pouch includes providing a pouch separate from a diaper for attachment to the diaper by the end consumer.
